(19) United States
(12) Plant Patent Application Publication
(10) Pub. No.: US 2021/0243928 P1
(43) Pub. Date: Aug. 5, 2021

(54) Raspberry Plant Named 'Yana'

(50) ;

(71) Applicant: EU PLANTS LTD, Finchampstead (GB)

(72) Inventor: Slavey Slavchev, Finchampstead (GB)

(21) Appl. No.: 17/164,322

(22) Filed: Feb. 1, 2021

(30) Foreign Application Priority Data
Jan. 30, 2020 (QZ) 2020/0331

(51) Int. Cl. A01H 6/74

(52) U.S. Cl. PLT/204

(57) ABSTRACT

Described is a new and distinct cultivar of red raspberry, Rubus ideas L., and referred herein by the variety denomination “YANA.”

### CROSS-REFERENCE TO RELATED APPLICATIONS

This application claims priority to and the benefit of European Union (EU) Community Plant Variety Application No. 2020/0331, filed Jan. 30, 2020; the entire contents of which as are hereby incorporated by reference herein.

Latin name of the genus and species: Rubus idaeus L.

Variety denomination: “YANA”

### BACKGROUND

Various embodiments of the present invention are directed toward a new and distinct cultivar of red raspberry, Rubus idaeus L., and referred herein by the variety denomination “YANA”. “YANA” was produced from seeds and unknown cross-breeding. More particularly, seeds were collected from fruit brought from British supermarkets in 2013 and were grown in the Summer of 2014 in Oxfordshire, England. The seeds pollinated in the Autumn in open fields. In 2015, 40,000 seeds produced as described were sown and produced 6,000 seedlings. These seedlings were crossed in the Spring of 2016 and one variety selected was collected and termed “YANA”. The resulting raspberry plants have been asexually reproduced repeatedly by in vitro propagation and by soft cuttings of shoots from the roots, since that time. The variety of the present invention has been found to retain its distinctive characteristics through successive asexual propagation in Oxfordshire, England.

### BRIEF SUMMARY

Generally described, various embodiments of the present invention exhibit multiple distinguishing characteristics.

The following are some of the distinguishing characteristics of this new cultivar variety of raspberry plant, YANA, when grown under normal horticultural practices in Oxfordshire, England:
Primocane raspberry plant.
Shape and size of leaves.
Exceptional fruit presentation.
Better fruit shape.
Better looks and better tastes than most Primocane varieties.
Better shelf-life and looks than known varieties.
Colour of the dormant cane.

### BRIEF DESCRIPTION OF THE PHOTOGRAPHS

The patent or application file contains at least one drawing executed in color. Copies of this patent or patent application publication with color drawing(s) will be provided by the Office upon request and payment of the necessary fee.

The new variety of raspberry plant, YANA is illustrated by the accompanying photographs which show the fruit of the plant as well as the Primocanes; the colours shown are true as according to conventional photographic procedures. The photographs in FIGS. 1 to 4 are of one-year old plants.

FIG. 1 shows the shape and dimensions of the fruit of YANA.

FIG. 2 shows the shape and conformation of YANA plants including leaf shape.

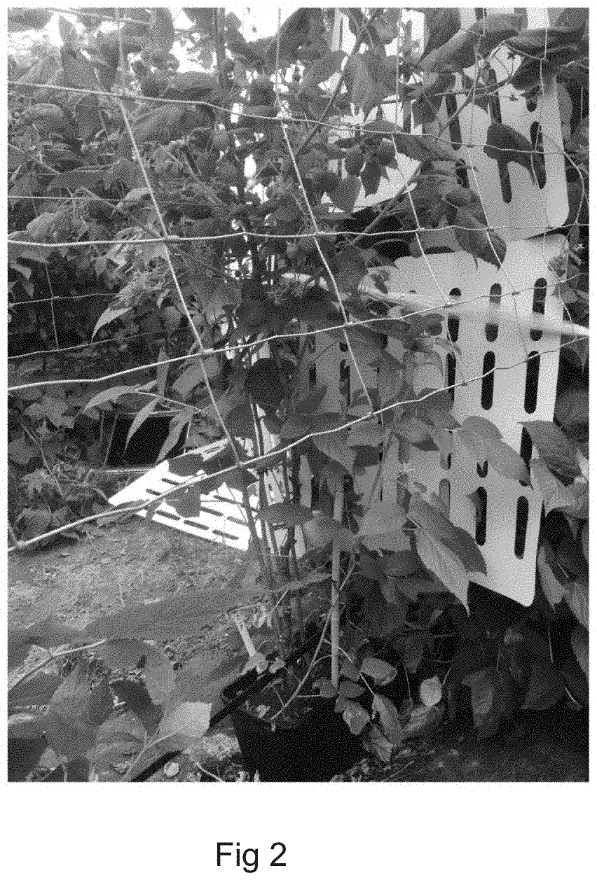

FIG. 3 shows the shape and conformation of YANA plants including leaf shape.

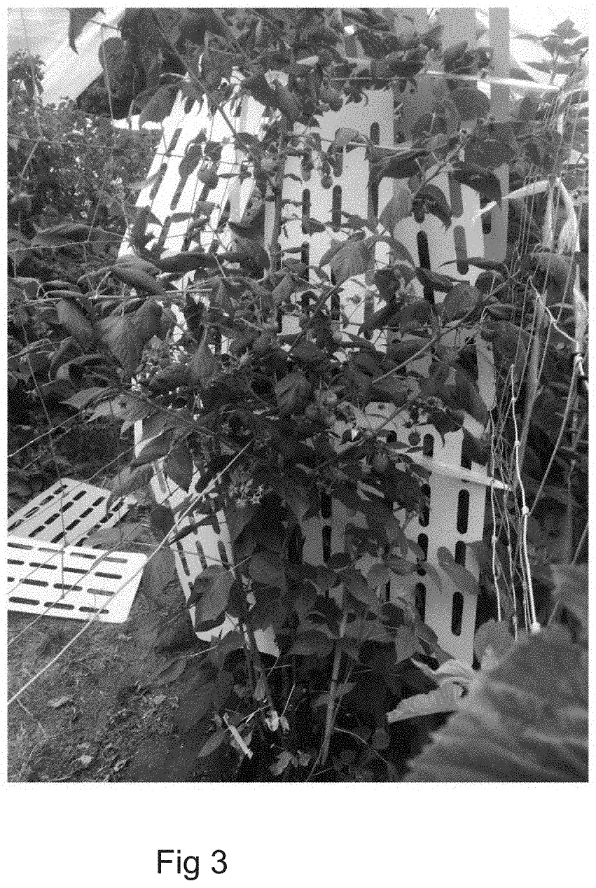

FIG. 4 shows the shape and conformation of the YANA plants.

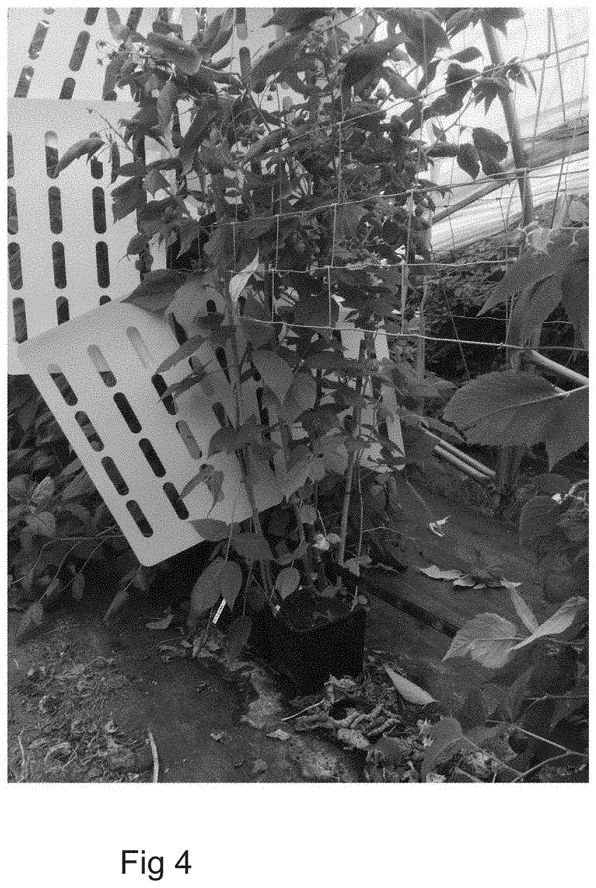

FIG. 5 shows the distinctive leaf shape of YANA (upper picture) and size compared to a typical commercial variety (lower picture).

FIG. 6 shows the typical size of the YANA raspberry fruit compared to a coin of about 27 mm.

FIG. 7 shows the nature of the cane colour and spine size and numbers of YANA.

### DETAILED BOTANICAL DESCRIPTION

Various embodiments of the present invention now will be described more fully hereinafter with reference to the accompanying drawings, in which some embodiments are shown. the embodiments described herein are provided so that this disclosure will satisfy applicable legal requirements.

The following description of “YANA” sets out distinctive characteristics of “YANA”. The description is based on UPVO terminology. Depending on environmental, seasonal, climatical or other conditions, the characteristics may vary in accordance with expected horticultural practice.

TABLE 1
I.

| Characteristics | YANA | Comparable varieties |
|---|---|---|
| Dormant cane colour | Brown | Caliber Glen Ova |
| Presence of spines | Present | Malling Promise |
| Fruit: ratio, | Medium | Glen Ova, |
| length/width | | Rafzeter |
| Fruit: general shape in | Broad conical | Malling Orion, |
| lateral view | | Meeker |
| Fruit: colour | Medium red | Glen Ova, |
| | | Malling Orion |
| Fruit: main bearing | Only on previous | Malling Promise |
| type | years cane in | |
| | Summer | |
| Time of beginning of | Early to medium | Glen Ova, |
| fruit ripening on | | Glen Moy, |
| previous years’ canes | | Rafzmach |

TABLE 2
II. Similar Varieties and Differences from these Varieties

| Denomination | Characteristic in | State of | State of |
|---|---|---|---|
| of similar | which the similar | expression of | expression |
| variety | variety is different | similar variety | of YANA |
| Kwanza | Droplets | Double droplets | Round droplets |
| Polka | Colour and taste | Less taste, darker | More taste, |
| | | colour | lighter colour |
| Diamond Jubilee | Droplets | Double droplets | Round droplets |
| Glen Amble | Shelf life | Short shelf life | Long shelf life |

III. Shelf-life testing

Raspberry fruit from YANA, and a variety of commercial varieties—Malling Bella, Malling Charm, Diamond Jubilee and Maravilla—to provide a practical commercial comparison were kept at room temperature 20-25 degrees, for 8 days. Malling Charm raspberries degraded most rapidly. The Diamond Jubilee Maravilla, YANA and Malling Bella raspberries lasted the same time before becoming too soft and mould starting to appear. The test was repeated with fresh fruit keeping the raspberry fruit in a fridge at 4 degrees. The same results were recorded after 10 days as with the ambient temperature tests. These tests show the good shelf life of YANA.

These results demonstrate that YANA has comparable shelf-life characteristics compared with current commercial raspberry varieties.

### IV. Taste

YANA produces sweet tasting raspberry fruit. It has relatively high sugar content, however it has a distinct strong raspberry taste. It is also very juicy which may be due to its size. Being bigger it holds more juice. It is very tasty in the sense of it having a classic raspberry taste and because it is very juicy it creates a very good balance.

V. Appearance of the fruit

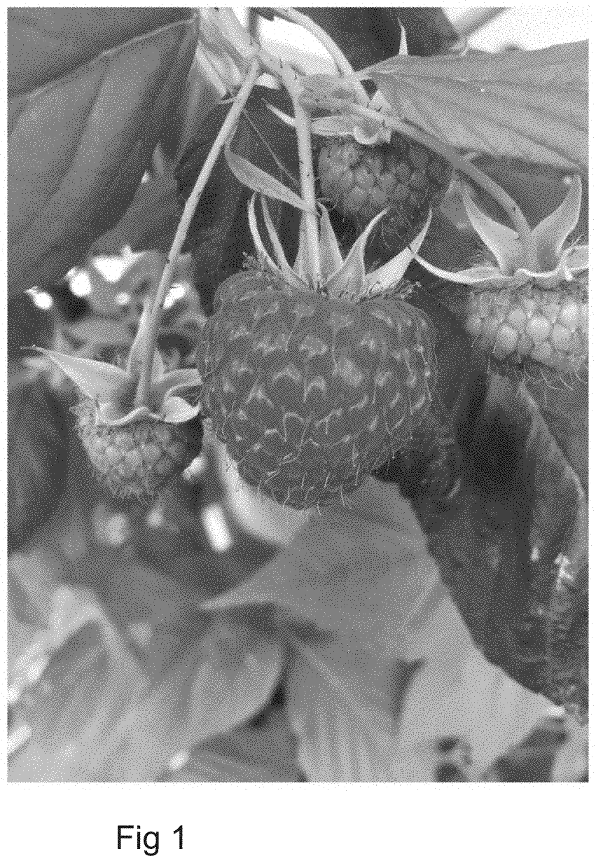

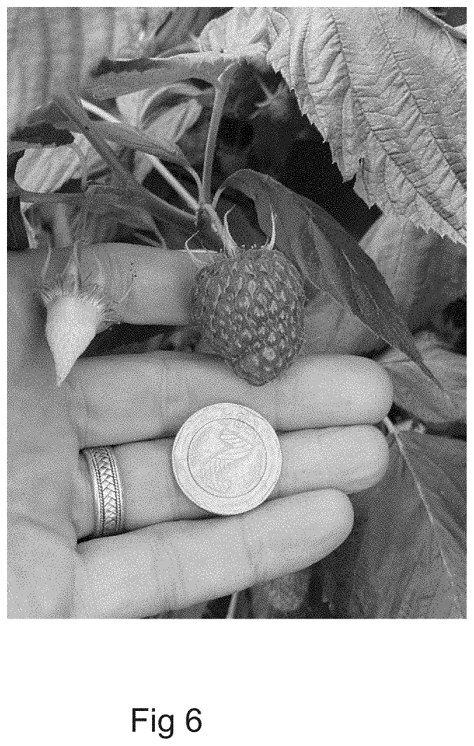

It has a quite unique and distinctive characteristic. The raspberries as shown particularly in in FIGS. 1 and 6 are particularly glossy in appearance. The single droplet being single probably contributes a lot the glossiness of the berry and YANA the glossiest berry known to the applicant.

VI. Appearance of the YANA raspberry plants

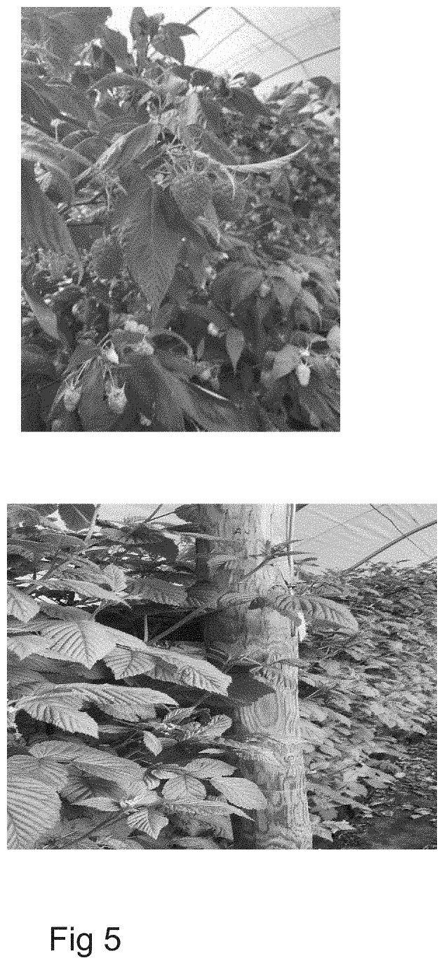

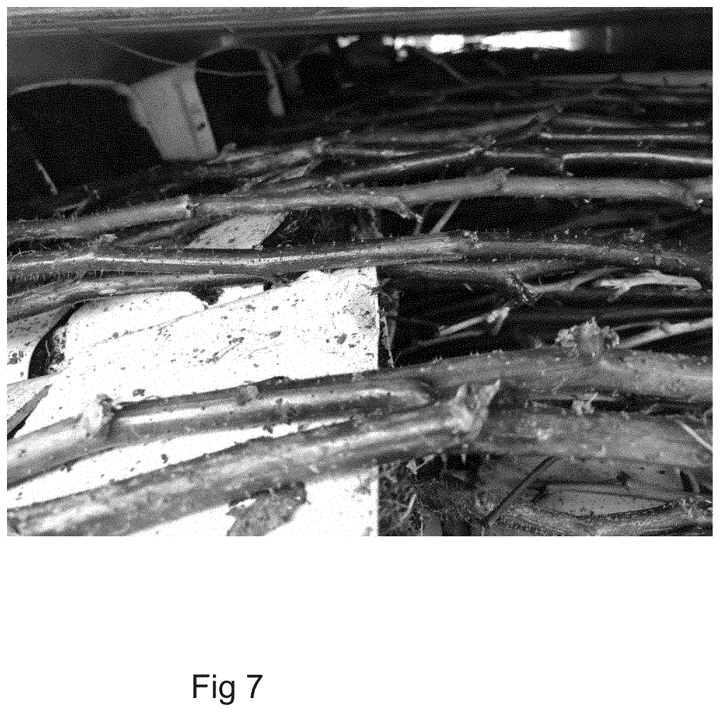

A combination of all its separate features makes YANA distinctive. The leaf shape and curve shown in the photographs are quite distinctive. The leaves are smaller than typical conventional varieties as shown in FIG. 5. It has relatively large spines as shown in FIG. 7. More, and bigger than Maravilla and DJ and Tulameen. The spines have a slight purplish tint to them. The slightly purplish brown of the dormant YANA cane is also a rare feature amongst raspberry plants. The root establishment of YANA is very good and quite quick, so it creates a dense root system that is also distinguishable from most other varieties. Its reproductive capabilities are also unseen with any other variety. It produces triple the tips from one root block in comparison to Maravilla for example.

Color(s) referenced herein and considered distinctive have been described and positively identified by a recognized color dictionary or color chart, namely The Horticultural Colour Chart of The Royal Horticultural Society, London, England (R.H.S. Colour Chart).

YANA grows well when pot grown in 100% coir substrate.

In terms of disease and insect resistance, “YANA” has been found to be resistant to Phytophthora and spider mite.

### V. Conclusion

Many modifications and other embodiments of the invention set forth herein will come to mind to one skilled in the art to which this invention pertains having the benefit of the teachings presented in the foregoing descriptions and the associated drawings. Although specific terms are employed herein, they are used in a generic and descriptive sense only and not for purposes of limitation.

That which is claimed:

1. A new and distinct cultivar of raspberry plant, YANA, substantially as illustrated and described, characterised by its fruit shape, leaf appearance and taste.
